(19) United States
(12) Patent Application Publication
(10) Pub. No.: US 2014/0045858 A1
(43) Pub. Date: Feb. 13, 2014

(54) COMPOUND PREPARATION TREATING ALZHEIMER'S DISEASE AND PREPARATION METHOD THEREOF

(71) Applicant: Yiming Wang, Beijing (CN)

(72) Inventor: Yiming Wang, Beijing (CN)

(21) Appl. No.: 14/008,837

(22) PCT Filed: Mar. 26, 2012
(86) PCT No.: PCT/CN2012/073025

§ 371 Date: Oct. 30, 2013

(30) Foreign Application Priority Data
Mar. 28, 2011 (CN) 201110074880.3

(51) Int. Cl. A61K 31/4965; A61K 31/4748

(52) U.S. Cl. CPC A61K 31/4965; A61K 31/4748
USPC 514/252.1

(57) ABSTRACT

A compound drug treating Alzheimer's disease, which is a transdermal delivery preparation mainly made by combining huperzine A and tetramethylpyrazine phosphate at a certain ratio. The compound preparation can protect the nervous system in synergy, and therefore can guard against and improve Alzheimer's disease from multiple targets.

### FIELD OF THE INVENTION

The present invention relates to a compound preparation treating Alzheimer's disease and a preparation method thereof, belonging to the field of medicine technology.

### BACKGROUND

Huperzine A, a new alkaloid extracted from Huperzia serrata, is a powerful, reversible and highly-selective acetylcholinesterase inhibitor, which has lasting effect as well as relatively small and reversible peripheral cholinergic side effects. Simultaneously, huperzine A can be seen as an effective drug for treating Alzheimer's disease due to its high fat solubility, small molecule, high possibility of passing through blood brain barriers, selective effect on central nervous system and stronger effect on cortex and hippocampal cholinesterase than on other cerebral sites. Two dosage forms of huperzine A are commercially available at present, i.e. tablet and capsule. It can be absorbed fast and completely by oral administration, broadly distributed in vivo and slowly metabolized out of the body, and has very high bioavailability.

Tetramethylpyrazine phosphate is the phosphate of ligustrazine. Ligustrazine is the main ingredient of the traditional Chinese medicine Ligusticum wallichii, widely applies to clinical treatment for cerebral thrombosis, cerebral arterial embolism, angina and other ischemic cardiovascular and cerebrovascular diseases, and has precise therapeutic effect and small toxic and side effects. As a novel calcium ion antagonist, ligustrazine has efficacies of dilating blood vessels, increasing coronary flow and cerebral blood flow, and improving calcium homeostasis disorder in nerve cells. In the meantime, ligustrazine is capable of eliminating free radicals, reducing lipid peroxidase in vivo, improving abnormal metabolism of free radicals, and plays a significant role in anti-aging and prevention for Alzheimer's disease. Currently, there are tetramethylpyrazine phosphate tablets, capsules and injections that are commercially available. Oral delivery of the tetramethylpyrazine phosphate, however, is followed by prominent liver first-pass effect, which is accompanied with short half life (t½=2.89h), therefore, its clinical application is limited to a certain extent.

Alzheimer's disease, a chronic neurodegenerative disease occurring at the senile or presenile stage, is pathologically featured by neurofibrillary tangles in nerve cells, senile plague caused by extracellular amyloid protein deposition, and neuronal loss. The drugs that have been relatively more applied in treating Alzheimer's disease so far are cholinesterase inhibitors, antioxidants, non-steroidal anti-inflammatory drugs, estrogens, calcium antagonists and drugs for improving cerebral metabolism. Plenty of pathogenesis is involved in Alzheimer's disease, and domestic and overseas studies have shown that Alzheimer's disease could derive directly or indirectly from such etiologies as cholinergic degeneration, inflammatory reaction, oxidative damage and apoptosis etc. Consequently, employment of a plurality of drugs for preventing and improving Alzheimer's disease from multiple targets has important significance.

### SUMMARY OF THE INVENTION

Given this, an objective of the present invention is to provide a new compound drug treating Alzheimer's disease and a preparation method thereof. The present invention adopts the compound preparation that is made from bulk drugs—huperzine A and tetramethylpyrazine phosphate by reasonable compatibility screening, and is capable of ameliorating the intracerebral cholinergic nerve function, resisting oxidative damage and improving the common symptoms in senile dementia patients from multiple targets to achieve the purpose of treating Alzheimer's disease effectively.

To achieve the aforementioned objective, the present invention provides a compound drug treating Alzheimer's disease. Said compound drug treating Alzheimer's disease is mainly made by combining two bulk drugs—huperzine A and tetramethylpyrazine phosphate. Said compound drug treating Alzheimer's disease is made as a transdermal delivery preparation, and its compositions in percentage by weight respectively are:

| | |
|---|---|
| huperzine A | 0.001-0.005%; |
| tetramethylpyrazine phosphate | 0.05-0.20%; |
| oil phase | 0.5-2%; |
| surfactant | 10-15%; |
| cosurfactant | 2-6%; |
| transdermal penentration enhancer | 0.5-2%; |
| polyvinyl pyrrolidone | 20.0-25.0%; |
| polyvinyl alcohol | 30.0-35.0%; |
| polyethylene glycol | 20.0-25.0%; |
| sorbitol | 3.0-5.0%; |

wherein, said oil phase is selected from one of the group consisting of or from combinations thereof: oleic acid, isopropyl myristate and medium chain triglycerides; said surfactant is selected from one of the group consisting of or from combinations thereof: polyoxyethylene ether-40 hydrogenated castor oil, poloxamer 188, and lecithin E-80; said cosurfactant is selected from one of the group consisting of or from combinations thereof: ethanol and 1, 2-propanediol; and said transdermal penetration enhancer is selected from one of the group consisting of or from combinations thereof: eucalyptus oil, D-limonene and turpentine.

A preparation method of the aforementioned compound drug treating Alzheimer's disease comprises the following steps:

(1) Homogeneously mixing the oil phase, the surfactant, the cosurfactant and the transdermal penetration enhancer at said ratio by stirring and then adding the huperzine A, and fully dissolving it to obtain a homogeneous and transparent solution;

(2) Weighing and adding the tetramethylpyrazine phosphate into water, fully dissolving the tetramethylpyrazine phosphate by ultrasonic treatment for 10-15 minutes, slowly adding the solution into the aforementioned solution, and homogeneously mixing them by continuous stirring to obtain huperzine A-tetramethylpyrazine phosphate microemulsion;

(3) Weighing a prescription amount of the polyvinyl alcohol, adding water and then carrying out stirring overnight, dissolving the polyvinyl alcohol by heating to 90° C., homogeneously mixing the polyvinyl alcohol with the water by stirring, and cooling the mixture to obtain a polyvinyl alcohol aqueous solution;

(4) Mixing the cooled polyvinyl alcohol aqueous solution, a polyvinyl pyrrolidone solution, a sorbitol aqueous solution and a fixed amount of water, and homogeneously stirring the mixture to obtain a homogeneous solution; and

(5) Slowly adding the huperzine A-tetramethylpyrazine phosphate microemulsion to the solution obtained in the step (4), homogeneously mixing them by stirring, coating the mixture on a flat plate, drying the mixture for 24 hours at 37° C., removing the dried mixture from the flat plate and cutting the dried mixture to obtain the transdermal delivery preparation.

Preferably, the solvent of the polyvinyl pyrrolidone solution in foregoing step (4) is prepared by mixing water with polyethylene glycol based on a volume ratio of 85:15.

The advantages of the present invention are: (1) huperzine A and tetramethylpyrazine phosphate _{playa} role of protecting the nervous system in synergy and can intervene the pathogenesis of Alzheimer's disease from multiple targets; (2) the transdermal delivery preparation is not viscous itself, before delivery, skin is coated with a fixed amount of water (15 μl.cm⁻²) and then covered by the patch to create viscosity, thus, the transdermal delivery preparation is convenient for delivery, simple in preparation process, easy in condition control, low in cost and applicable for mass production; and (3) the transdermal delivery preparation could increase the solubility of the fat-soluble drug Huperzine A. avoid the liver first-pass effect of the tetramethylpyrazine phosphate, shorten the delivery interval to a large extent, improve the compliance of patients and intervene different pathogenic pathways of Alzheimer's disease, and it has important significance for treating Alzheimer's disease from multiple targets.

Specific Embodiments

For further disclosure of the features and technical contents of the present invention, reference will be made to the detailed description and accompanying drawings that are related to the present invention below, however, the accompanying drawings are given for reference and description only and not intended to limit the present invention.

The present invention will be further described below with reference to the specific examples.

### Example 1

### Preparation of the Transdermal Delivery System

After 0.2 g oleic acid (which is used as oil phase and is a substrate for microemulsion's formation), 2.4 g polyoxyethylene ether-40 hydrogenated castor oil (which is used as a cosurfactant to promote microemulsion's formation), 0.8 g ethanol (which is used as a cosurfactant to promote microemulsion's formation and increase its stability) and 0.2 g eucalyptus oil (which is used as a transdermal penetration enhancer to promote and assist better penetration of the drug into skin) are homogeneously mixed by stirring, 0.008 g huperzine A is added and fully dissolved to obtain a homogeneous and transparent solution; 1.8 g tetramethylpyrazine phosphate is weighed, added into water, and fully dissolved by ultrasonic treatment for 10-15 minutes, the solution is slowly added into the aforementioned solution and is homogeneously mixed by continuous stirring to obtain huperzine A-tetramethylpyrazine phosphate microemulsion; 112 g polyvinyl alcohol (PVA) (which is used as an adhesive for the preparation) is weighed, water is added and stirred overnight, the polyvinyl alcohol is dissolved by heating to 90° C. and then homogeneously mixed with the water by stirring, and the mixture is cooled to obtain a 10% PVA solution; 103 g polyvinyl pyrrolidone (PVP) (which is used as a viscosity increaser for the preparation) is weighed and water is added: PEG (the volume ratio is 85:15) solution (wherein, the PEG serves as a moisturizing agent), and dissolution is achieved by stirring to obtain a 21% PVP solution; 51 g sorbitol is weighed and added with water for its dissolution, so as to obtain a 70% sorbitol aqueous solution (wherein, the sorbitol serves as a moisturizing agent); the cooled 10% PVA solution, the 21% PVP solution, the 70% sorbitol aqueous solution and a fixed amount of water are mixed and stirred homogeneously, the huperzine A-tetramethylpyrazine phosphate microemulsion is added to the solution and homogeneously mixed with the solution by stirring, the resulting mixture is coated on a flat plate, dried for 24 hours at 37° C., removed from the flat plate and cut to obtain 100 transdermal delivery preparations in a patch form, namely, the compound drugs.

### Example 2

### Preparation of the Transdermal Delivery System

After 0.2 g oleic acid (which is used as oil phase and is a substrate for microemulsion's formation), 2.4 g polyoxyethylene ether-40 hydrogenated castor oil (which is used as a cosurfactant to promote microemulsion's formation), 0.8 g ethanol (which is used as a cosurfactant to promote microemulsion's formation and increase its stability) and 0.2 g eucalyptus oil (which is used as a transdermal penetration enhancer to promote and assist better penetration of the drug into skin) are homogeneously mixed by stirring, 0.0096 g huperzine A is added, and fully dissolved to obtain a homogeneous and transparent solution; 14.41 g tetramethylpyrazine phosphate is weighed, added into water, and is fully dissolved by ultrasonic treatment for 10-15 minutes, the solution is slowly added into the aforementioned solution and is homogeneously mixed by continuous stirring to obtain huperzine A-tetramethylpyrazine phosphate microemulsion; 112 g polyvinyl alcohol (PVA) (which is used as an adhesive for the preparation) is weighed, water is added and stirred overnight, the polyvinyl alcohol is dissolved by heating to 90° C. and then homogeneously mixed with the water by stirring, and the mixture is cooled to obtain a 10% PVA solution; 103 g polyvinyl pyrrolidone (PVP) (which is used as a viscosity increaser for the preparation) is weighed and water is added: PEG (the volume ratio is 85:15) solution (wherein, the PEG serves as a moisturizing agent), and dissolution is achieved by stirring to obtain a 21% PVP solution; 51 g sorbitol is weighed and added with water for its dissolution, so as to obtain a 70% sorbitol aqueous solution (wherein, the sorbitol serves as a moisturizing agent); the cooled 10% PVA solution, the 21% PVP solution, the 70% sorbitol aqueous solution and a fixed amount of water are mixed and stirred homogeneously, the huperzine A-tetramethylpyrazine phosphate microemulsion is added to the solution and homogeneously mixed with the solution by stirring, the resulting mixture is coated on a flat plate, dried for 24 hours at 37° C., removed from the flat plate and cut to obtain 100 transdermal delivery preparations in a patch form, namely, the compound drugs.

### Example 3

### Preparation of the Transdermal Delivery System

After 0.2 g oleic acid (which is used as oil phase and is a substrate for microemulsion's formation), 2.4 g polyoxyethylene ether-40 hydrogenated castor oil (which is used as a cosurfactant to promote microemulsion's formation), 0.8 g ethanol (which is used as a cosurfactant to promote microemulsion's formation and increase its stability) and 0.2 g eucalyptus oil (which is used as a transdermal penetration enhancer to promote and contribute to better penetration of the drug into skin) are homogeneously mixed by stirring, 0.012 g huperzine A is added, and fully dissolved to obtain a homogeneous and transparent solution; 0.9 g tetramethylpyrazine phosphate is weighed, added into water, and is fully dissolved by ultrasonic treatment for 10-15 minutes, the solution is slowly added into the aforementioned solution and is homogeneously mixed by continuous stirring to obtain huperzine A-tetramethylpyrazine phosphate microemulsion; 112 g polyvinyl alcohol (PVA) (which is used as an adhesive for the preparation) is weighed, water is added and stirred overnight, the polyvinyl alcohol is dissolved by heating to 90° C. and than homogeneously mixed with the water by stirring, and the mixture is cooled to obtain a 10% PVA solution; 103 g polyvinyl pyrrolidone (PVP) (which is used as a viscosity increaser for the preparation) is weighed and water is added: PEG (the volume ratio is 85:15) solution (wherein, the PEG serves as a moisturizing agent), and dissolution is achieved by stirring to obtain a 21% PVP solution; 51 g sorbitol is weighed and added with water for its dissolution, so as to obtain a 70% sorbitol aqueous solution (wherein, the sorbitol serves as a moisturizing agent); the cooled 10% PVA solution, the 21% PVP solution, the 70% sorbitol aqueous solution and a fixed amount of water are mixed and stirred homogeneously, the huperzine A-tetramethylpyrazine phosphate microemulsion is added to the solution and homogeneously mixed with the solution by stirring, the resulting mixture is coated on a flat plate, dried for 24 hours at 37° C., removed from the flat plate and cut to obtain 100 transdermal delivery preparations in a patch form, namely, the compound drugs.

### Example 4

### Preparation of the Transdermal Delivery System

After 0.1 g isopropyl myristate (which is used as oil phase and is a substrate for microemulsion's formation), 2.0 g lecithin E-80 (which is used as a surfactant to promote microemulsion's formation), 0.4 g 1, 2-propanediol (which is used as a cosurfactant to promote microemulsion's formation and increase its stability) and 0.1 g D-limonene (which is used as a transdermal penetration enhancer to promote and contribute to better penetration of the drug into skin) are homogeneously mixed by stirring, 0.004 g huperzine A is added, and fully dissolved to obtain a homogeneous and transparent solution; 0.9 g tetramethylpyrazine phosphate is weighed, added into water, and is fully dissolved by ultrasonic treatment for 10-15 minutes, the solution is slowly added into the aforementioned solution and is homogeneously mixed by continuous stirring to obtain huperzine A-tetramethylpyrazine phosphate microemulsion; 101 g polyvinyl alcohol (PVA) (which is used as an adhesive for the preparation) is weighed, water is added and stirred overnight, the polyvinyl alcohol is dissolved by heating to 90° C. and then homogeneously mixed with the water by stirring, and the mixture is cooled to obtain a 10% PVA solution; 93 g polyvinyl pyrrolidone (PVP) (which is used as a viscosity increaser for the preparation) is weighed and water is added: PEG (the volume ratio is 85:15) solution (wherein, the PEG serves as a moisturizing agent), and dissolution is achieved by stirring to obtain a 21% PVP solution; 38 g sorbitol is weighed and added with water for its dissolution, so as to obtain a 70% sorbitol aqueous solution (wherein, the sorbitol serves as a moisturizing agent); the cooled 10% PVA solution, the 21% PVP solution, the 70% sorbitol aqueous solution and a fixed amount of water are mixed and stirred homogeneously, the huperzine A-tetramethylpyrazine phosphate microemulsion is added to the solution to obtain a solution B; the solution B is homogenously mixed by stirring, coated on a flat plate, dried for 24 hours at 37° C., removed from the flat plate and cut to obtain 100 transdermal delivery preparations in a patch form, namely, the compound drugs.

### Example 5

### Preparation of the Transdermal Delivery System

After 0.4 g medium chain triglycerides (which is used as oil phase and is a substrate for microemulsion's formation), 3.09 poloxamer 188 (which is used as a surfactant to promote microemulsion's formation), 1.2 g ethanol (which is used as a cosurfactant to promote microemulsion's formation and increase its stability) and 0.4 g turpentine (which is used as a transdermal penentration enhancer to promote and contribute to better penetration of the drug into skin) are homogeneously mixed by stirring, 0.02 g huperzine A is added, and fully dissolved to obtain a homogeneous and transparent solution; 3.6 g tetramethylpyrazine phosphate is weighed, added into water, and fully dissolved by means of ultrasonic treatment for 10-15 minutes, the solution is slowly added into the aforementioned solution and is homogeneously mixed by continuous stirring to obtain huperzine A-tetramethylpyrazine phosphate microemulsion; 123 g polyvinyl alcohol (PVA) (which is used as an adhesive for the preparation) is weighed, water is added and stirred overnight, the polyvinyl alcohol is dissolved by heating to 90° C. and then homogeneously mixed with the water by stirring, and the mixture is cooled to obtain a 10% PVA solution; 117 g polyvinyl pyrrolidone (PVP) (which is used as a viscosity increaser for the preparation) is weighed and added with a water: PEG (the volume ratio is 85:15) solution (wherein, the PEG serves as a moisturizing agent), and dissolution is achieved by stirring to obtain a 21% PVP solution; 64 g sorbitol is weighed and added with water for its dissolution, so as to obtain a 70% sorbitol aqueous solution (wherein, the sorbitol serves as a moisturizing agent); the cooled 10% PVA solution, the 21% PVP solution, the 70% sorbitol aqueous solution and a fixed amount of water are mixed and stirred homogeneously, the huperzine A-tetramethylpyrazine phosphate microemulsion is added to the solution and homogeneously mixed with the solution by stirring, the resulting mixture is coated on a flat plate, dried for 24 hours at 37° C., removed from the flat plate and out to obtain 100 transdermal delivery preparations in a patch form, namely, the compound drugs.

A pharmacodynamic study on the compound drug of the present invention and its results are given below:

The study employs 56 rats which are randomly divided into 7 groups, with 8 rats in each group. Group 1 is considered as the model group, in which blank patches are delivered to the rats; Group 2 is considered as the blank control group, in which normal saline is delivered to the rats in an intragastric infusion way once a day; Groups 3-7 are respectively delivered with transdermal delivery preparations containing different doses: Group 3 contains 6.70×10⁻² mg/kg of huperzine A and 2.26 mg/kg of tetramethylpyrazine phosphate (Example 1); Group 4 contains 8.04×10⁻² mg/kg of huperzine A and 18.1 mg/kg of tetramethylpyrazine phosphate (Example 2); Group 5 contains 10.05×18⁻²mg/kg of huperzine A and 1.13 mg/kg of tetramethylpyrazine phosphate (Example 3); Group 6 contains 6.70×10⁻² mg/kg of huperzine A and Group 7 contains 2.26 mg/kg of tetramethylpyrazine phosphate, to serve as Single-Component Control Groups. The patches are delivered to the rats once every three days and this delivery lasts for 9 days. Scopolamine hydrobromide (1.5 mg/kg) is delivered to the rats in an intraperitoneal injection way on the tenth day in order to establish a rat amnesia model, the rats are executed by breaking their necks 30 minutes after intraperitoneal injection, their brains are taken out and cleaned with saline, the moisture is thoroughly absorbed by filter paper, and these brains are weighed and put in refrigerator with a temperature of −80° C. for future use. While measuring, 10% brain tissue homogenate is prepared under an ice bath by 0.9% sodium chloride solution and then centrifuged for 15 minutes at a speed of 3500 rpm at 4° C., the supernatant is collected, the activities/contents of acetylcholin esterase (AChE), superoxide dismutase (SOD), glutathione peroxidase (GSH-Px) and malondialdehyde (MDA) in the homogenate are measured in accordance with kit's operation steps, and experimental results are shown in Table 1.

TABLE 1
Results of the Pharmacodynamic Study (n = 8, χ̅ ± s)

| | Ache | SOD | GSH-Px | MDA |
|---|---|---|---|---|
| | (U/mg | U/mg | (U/mg | (nmol/mg |
| Group | protein) | protein) | protein) | protein) |
| Model | 1.48 ± 0.11 | 54.23 ± 2.77 | 76.50 ± 3.78 | 6.92 ± 0.55 |
| Group | | | | |
| Blank | 0.78 ± 0.04 | 84.12 ± 3.56 | 104.58 ± 5.98 | 3.96 ± 0.21 |
| Control | | | | |
| Group | | | | |
| Group 3 | 0.84 ± 0.05* | 83.33 ± 2.54* | 99.89 ± 6.28* | 4.08 ± 0.45* |
| Group 4 | 0.81 ± 0.05* | 83.69 ± 2.70* | 102.01 ± 7.15* | 4.06 ± 0.51* |
| Group 5 | 0.79 ± 0.04* | 83.53 ± 2.84* | 98.54 ± 4.03* | 4.05 ± 0.42* |
| Group 6 | 0.89 ± 0.07* | 70.47 ± 2.05* | 90.36 ± 5.34* | 4.81 ± 0.46* |
| Group 7 | 1.17 ± 0.08* | 80.37 ± 3.28* | 95.32 ± 5.68* | 4.27 ± 0.41* |

Note:
each of the delivery groups (Groups 3-7) compared with model group,
*P < 0.01; model group compared with blank control group, cted
**P < 0.01.

The results in Table 1 show that: first of all, when a comparison between the model group and the blank control group is conducted, the activity of AChE in the model group increases significantly (P <0.001); the activities of SOD and GSH-Px decrease significantly (P <0.001); and at the same time, the content of MDA increases significantly (P <0.001), this indicates that rats' cholinergic and oxidative systems are both damaged after delivery of scopolamine, which means successful modeling. Secondly, the experimental results of the Group 3 as well as of two single-component control groups, i.e. the Group 6 (single component: huperzine A) and the Group 7 (single component: tetramethylpyrazine phosphate), show that: huperzine A has not only certain function of anti-cholinergic damage, but also certain effect of anti-oxidation; tetramethylpyrazine phosphate has a very strong anti-oxidation effect, but relatively poor function of anti-cholinergic damage. But a drug that contains both huperzine A and tetramethylpyrazine phosphate at the same time is able to significantly reduce the activity of AChE, raise the activities of SOD and GSH-Px and reduce the content of MDA, as a result, huperzine A and tetramethylpyrazine phosphate have a synergetic effect. Finally, the activities/contents of AChE, SOD, GSH-Px and MDA in Groups 3-5 approximate to those in the blank control group, that is to say, the levels of oxidase in rat's cholinergic and oxidative systems in the various delivery groups are close to normal level.

To sum up, the huperzine A/tetramethylpyrazine phosphate-containing compound preparation provided in the present invention is capable of intervening the pathogenesis of Alzheimer's disease from multiple targets, achieving the effects of prevention and treatment.

Discussed above are the listed preferred embodiments of the present invention only, which are not intended to limit the scope of the present invention. Any equivalent modifications or variations made by ordinary skilled in this art using the present invention shall likewise fall within the scope of patent protection of the present invention.

What is claimed is:

1. A compound drug treating Alzheimer's disease, which is mainly made by combining two bulk drugs—huperzine A and ligustrazine phosphate, characterized in that, said compound drug treating Alzheimer's disease is made as a transdermal delivery preparation, and its compositions in percentage by weight respectively are:

| | |
|---|---|
| huperzine A | 0.001-0.005%; |
| ligustrazine phosphate | 0.05-0.20%; |
| oil phase | 0.5-2%; |
| surfactant | 10-15%; |
| cosurfactant | 2-6%; |
| transdermal penentration enhancer | 0.5-2%; |
| polyvinyl pyrrolidone | 20.0-25.0%; |
| polyvinyl alcohol | 30.0-35.0%; |
| polyethylene glycol | 20.0-25.0%; |
| sorbitol | 3.0-5.0%; |

wherein, said oil phase is selected from one of the group consisting of or from combinations thereof: oleic acid, isopropyl myristate and medium chain triglycerides; said surfactant is selected from one of the group consisting of or from combinations thereof:
polyoxyethylene ether-40 hydrogenated castor oil, poloxamer 188, and lecithin E-80; said cosurfactant is selected from one of the group consisting of or from combinations thereof:
ethanol and 1, 2-propanediol; and said transdermal penetration enhancer is selected from one of the group consisting of or from combinations thereof: eucalyptus oil. D-limonene and turpentine.

2. A preparation method of the compound drug according to claim 1, characterized in that, the preparation method comprises the following steps:
(1) Homogeneously mixing the oil phase, the surfactant, the cosurfactant and the transdermal penetration enhancer at said ratio by stirring and then adding the huperzine A, and fully dissolving it to obtain a homogeneous and transparent solution;
(2) Weighing and adding the ligustrazine phosphate into water, fully dissolving the ligustrazine phosphate by ultrasonic treatment for 10-15 minutes, slowly adding the solution into the aforementioned solution, and homogeneously mixing them by continuous stirring to obtain huperzine A-ligustrazine phosphate microemulsion;
(3) Weighing a prescription amount of the polyvinyl alcohol, adding water and then carrying out stirring overnight, dissolving the polyvinyl alcohol by heating to 90° C., homogeneously mixing them by stirring, and cooling the mixture to obtain a polyvinyl alcohol aqueous solution;
(4) Mixing the cooled polyvinyl alcohol aqueous solution, a polyvinyl pyrrolidone solution, a sorbitol aqueous solution and a fixed amount of water, and homogeneously stirring the mixture to obtain a homogeneous solution; and
(5) Slowly adding the huperzine A-ligustrazine phosphate microemulsion to the solution obtained in the step (4), homogeneously mixing them by stirring, coating the mixture on a flat plate, drying the mixture for 24 hours at 37° C., removing the dried mixture from the flat plate and cutting the dried mixture to obtain the transdermal delivery preparation.

3. The preparation method according to claim 2, characterized in that, the solvent of the polyvinyl pyrrolidone solution is prepared by mixing water with polyethylene glycol based on a volume ratio of 85:15.
